(19) United States
(12) Patent Application Publication
(10) Pub. No.: US 2025/0235681 A1
(43) Pub. Date: Jul. 24, 2025

(54) ULTRASONIC PROBE TO IMPROVE ABSORPTION OF ACTIVE INGREDIENTS INTO THE SKIN

(71) Applicant: Dyson Technology Limited, Wiltshire (GB)

(72) Inventor: Benjamin John KNIGHT-GREGSON, Bristol (GB)

(21) Appl. No.: 18/697,818

(22) PCT Filed: Oct. 4, 2022
(86) PCT No.: PCT/GB2002/052507

§ 371 Date: Apr. 2, 2024

(30) Foreign Application Priority Data
Oct. 13, 2021 (GB) 2114613.9

(51) Int. Cl. A61M 37/00; A45D 34/04

(52) U.S. Cl. CPC A61M 37/0092; A45D 34/041; A45D 2200/207; A61M 2037/0007

(57) ABSTRACT

An ultrasonic probe is provided and includes a rollable probe head and an ultrasonic transducer. The rollable probe head is configured to couple sound energy emitted from the ultrasonic transducer to a working surface. The ultrasonic probe further includes a reservoir for containing a flowable substance. The rollable probe head is configured to dispense the flowable substance from the reservoir to the working surface when rotated. The probe head is configured to roll smoothly across a working surface, such as skin, smoothly whilst delivering an appropriate amount of flowable substance to the skin as it rolls.

### FIELD OF THE INVENTION

The invention relates generally to an ultrasonic device or probe, particularly although not exclusively, of the type that are used to increase the uptake or absorption of active compounds and ingredients in cosmetic products that are applied to the surface of skin, such as creams and gels.

### BACKGROUND

Ultrasonic devices are known for use in laboratory settings for processing biological samples. Such devices emit ultrasonic energy which induces cavitation in the sample, thereby agitating the particles in the sample to achieve certain objectives. For example, it is known to use ultrasonic transducers/probes for waste-water purification, antioxidant extraction, cell disruption for DNA analysis and nanoparticle production.

More recently, lower power ultrasonic devices have emerged in the personal care field which are marketed as products that apply ultrasonic energy to the skin to benefit the absorption of active compounds into the skin. Examples of ultrasonic devices used for these purposes are disclosed in U.S. Pat. No. 7,427,273 and US2004010222.

One limitation of the use of ultrasonic devices as personal care devices is that the cooling requirements of such devices can mean that the devices have limited run time. Measures to limit device temperature are to use a lower power level, which can reduce the effectiveness of the device. Furthermore, the probe heads or tips used for known ultrasonic devices tend to have a generally flat configuration which have drawbacks in terms of their manoeuvrability, in use.

It is against this background that the invention has been devised.

### SUMMARY OF THE INVENTION

According to a first aspect of the invention, there is provided an ultrasonic probe comprising a rollable probe head and an ultrasonic transducer, wherein the rollable probe head is configured to couple sound energy emitted from the ultrasonic transducer to a working surface, in use. The ultrasonic probe further comprises a reservoir for containing a flowable substance, and wherein the rollable probe head is configured to dispense the flowable substance from the reservoir to the working surface when rotated.

A benefit of the invention is that probe head is able to roll smoothly across a working surface, such as skin, smoothly whilst delivering an appropriate amount of flowable substance to the skin as it rolls. This avoids the need to apply a gel, cream or aqueous carrier directly to the skin prior to using the probe. The flowable substance is therefore used more sparingly and, thus, more efficiently. A further advantage is that the flowable substance can, in some embodiments, serve a further purpose in providing a cooling medium for the ultrasonic transducer which may be positioned within the probe so as to be at least partly immersible in the flowable substance, which is therefore able to act as a coolant.

At least part of the rollable probe head may be located in an outlet of the reservoir. The probe may be circular, like a wheel or a barrel, such that the rollable probe head is rotatable about a single rolling axis. In a preferred embodiment, the rollable probe head is generally spherical in form. This configuration helps the probe head roll in different directions. In this sense, it may be considered to have multiple rolling axes.

The position and configuration of the rollable probe head and the surrounding outlet may be complementarily shaped to define a gap therebetween to allow the flowable substance to exude through the gap. A flow control arrangement may be provided to increase the resistance to the flow of the flowable substance. For example, a porous strip in the form of a ring of reticulated foam rubber material may be provided in the outlet as a means of flow control. Foam allows a limited amount of flowable substance to pass through the outlet and may be configured to allow a predetermined amount of flow. The flow control arrangement may also be embodied as a plurality of discrete channels.

In an alternative embodiment, the ultrasonic transducer may be located inside the rollable probe head. Usefully, this means that the transducer is located more closely to the working surface which can improve the acoustic coupling.

In the case where the ultrasonic transducer is inside the rollable probe head, the transducer may be part of a transducer package including an integrated power source. This simplifies the provision of power to the transducer as it removes the need to pass a power supply through the rollable probe head because it is self-contained.

In order to ensure that the ultrasonic transducer remains in a suitable orientation to direct acoustic energy to the working surface, embodiments of the invention provide for the transducer to be supported inside the rollable probe head by a gimbal frame that permits the ultrasonic transducer to move relative to the rollable probe head. Advantageously, therefore, the transducer is able to move relative to the probe head and remain in an orientation to direct acoustic energy towards the skin. Such a configuration would benefit from being self-righting, as might be achieved by a suitable mass placement such that the centre of gravity is in a relatively lower position relative to the geometric centre of the rollable probe head.

The gimbal frame may take different forms. One option, for example, is for the gimbal frame to be suspended inside the rollable probe head by a plurality of bracing points that bear against an inside surface of the rollable probe head and permit relative movement of the gimbal frame. In this way, the gimbal frame is a relatively open frame assembly composed of struts or members spaced within the rollable probe head which is able to rotate inside of the rollable probe head.

As an alternative, the gimbal frame may be configured such that it is supported inside the rollable probe head by a fluid support. For example, the fluid support may include a housing encircling the transducer and which defines a fluid-filled gap between the housing and the rollable probe head. Such a configuration may take the form of ‘nested spheres’ wherein the gap between the spheres or balls is filled with a fluid which allows the two parts to rotate with respect to each other.

In examples where the ultrasonic transducer is located within the rollable probe head, the probe head may contain couplant fluid to facilitate transmission of energy emitted from the ultrasonic transducer out of the rollable probe head. In such a configuration, the ultrasonic transducer may at least partially be immersed in the couplant fluid such that the ultrasonic transducer is cooled by the couplant fluid. This configuration improves the cooling of the transducer and therefore can benefit the runtime of the probe.

In other examples of the invention, the ultrasonic transducer may be located remote from the rollable probe head. Such a configuration provides easier access to the transducer as it is outside of the rollable probe head. Packaging of the transducer is therefore less constrained which may provide cost and size advantages. In one arrangement, the rollable probe head is located between the working surface and the ultrasonic transducer. The ultrasonic transducer therefore emits acoustic energy through and out of the rollable probe head into the working surface. In some examples, the ultrasonic transducer may be located in the reservoir such that it is immersible in the flowable substance.

It should be noted that in the specific examples of the invention discussed here, the probe is not being used as a receiver to receive ultrasonic echoes generated by the transducer, although it is envisaged that such usage is not excluded. In this sense, therefore, the probe can be considered an ultrasonic applicator.

In another aspect, the invention provides an ultrasonic cosmetic probe for applying acoustic energy to skin, the probe comprising a rollable probe head and an ultrasonic transducer, wherein the rollable probe head is configured to couple sound energy emitted from the ultrasonic transducer to a skin surface. Preferred and/or optional features of the previous aspects of the invention also apply to this aspect of the invention. Thus, even without the ability to dispense a flowable substance to a working surface, the probe of the invention provides an improved probe or applicator device for the specific use of applying cosmetic treatment products to a skin surface. The probe of the invention is able to roll smoothly over the skin surface which reduces friction and so does not provide a feeling of ‘dragging’ across the skin. It also improves the ability of the probe to follow a contour of the skin, for example around bony protrusions such as jaw lines.

Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

The above and other aspects of the invention will now be described, by way of example only, with reference to the accompanying drawings, in which:

FIG. 1 is a schematic view of an ultrasonic probe in accordance with an example of the invention;

FIG. 2 is a schematic view of an ultrasonic probe in accordance with another example of the invention;

FIG. 3 is a schematic view of an ultrasonic probe, similar to that of FIG. 2, and in accordance with another example of the invention;

FIG. 4 is a schematic view of an ultrasonic probe, similar to that of FIG. 2, and in accordance with another example of the invention;

FIGS. 5a to 5c show various views of another example of the invention;

FIG. 6 is a schematic view of a further example of the invention.

In the drawings, the same reference numerals are used to denote features that are common across drawings.

### SPECIFIC DESCRIPTION

The invention relates to a configuration of an ultrasonic probe, wand or device which has particular utility in the field of personal care where it can be used on exposed skin to encourage the absorption of active compounds, such as moisturising ingredients, anti-aging products, analgesics, and non-steroidal anti-inflammatory creams, to name a few examples. As will be appreciated from this discussion, the configuration of the probe is such that it has improved manoeuvrability when used on skin since it can roll smoothly and can therefore be directed with precision and without excessive rubbing on the skin surface. Moreover, it is configured to dispense a flowable substance directly onto the skin surface as it rolls, which is particularly convenient as it avoids the need to apply that substance separately. This is particularly beneficial for flowable substances containing active ingredients which are expensive, as it permits a sufficient amount of substance to be deposited on the skin whilst avoiding wastage. A further advantage is that because the flowable substance is deposited as the device is moved over the skin, the substance creates a trail which indicates its route. This may be useful in terms of treating the skin surface evenly, compared to the known technique of depositing an excessive amount of e.g. moisturiser on the skin surfacer and then moving that substance around without any indication of coverage.

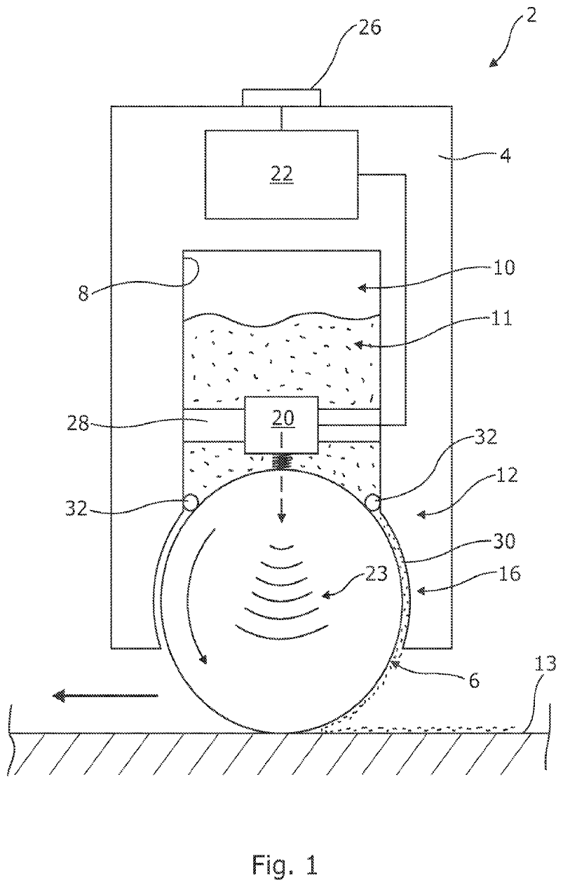

With reference to FIG. 1, an ultrasonic probe or wand 2 in accordance with the invention comprises a housing 4 and a probe head 6. Since FIG. 1 is schematic, the housing 4 is shown in a basic functional form and not styled for aesthetics or ergonomics.

The housing 4 defines an internal cavity 8 that acts as a reservoir 10 for a flowable substance 11, such as a gel, cream or aqueous solution. The internal cavity 10 may take any form within the housing 4 and the illustrated shape should not be considered limiting.

The cavity 8 terminates in an opening or outlet 12 which is shown at a lower end of the housing 4. The probe head 6 is rollable and is supported in the opening 12 so that it is able to rotate with respect to the housing 4. As shown in FIG. 1, the probe 2 is shown oriented with respect to a working surface 13 so that it is generally upright, with the rollable probe head 6 in contact with the working surface 13.

The rollable probe head 6 may take different forms. Here, it is shown as generally spherical in form. However, it may also be in the form of a cylindrical wheel or barrel.

Usefully, the spherical shape of the rollable probe head 6, in the illustrated example, means that it is able to rotate multi-axially with respect to the housing 4 so that the probe 2 can be moved across the working surface 13 in different directions with ease. In this respect, the opening 12 is annular in form and is shaped to define a flared receiving section 16 which is shaped to accommodate the rollable probe head 6 within it. In this respect, the receiving section 16 can be considered to define a part-spherical chamber. The receiving section 16 is further configured to retain the rollable probe head 6 within it so that it cannot fall from the outlet. This can be achieved in various ways. For example, the receiving section 16 can extend downwards past the mid-point of the rollable probe head 6 to where it narrows, thereby holding it in place. Alternatively or additionally, a retaining structure (not shown) can be added to the receiving section 16 to ensure that the rollable probe head remains in place and cannot fall out.

In other embodiments, it is envisaged that the rollable probe head 6 may have a spherical shape but is provided with an axle (not shown) through which it is mounted to the housing and therefore is constrained with a single axis of rotation.

The probe 2 also comprises an ultrasonic transducer 20 and a control system 22 for providing control signals to the ultrasonic transducer 20 through a suitable connection 24. The control system 22 is operated by a user interface 26. The user interface 26 may take various forms, for example one or more user-operable hard-wired buttons, to a touch-sensitive display screen. The precise form of user interface is not central to the invention and so will not be described in further detail. A suitable power source is not shown, but its presence is implied. The ultrasonic transducer 20 is operable to emit acoustic energy at suitable frequency levels and energy levels required for the purpose of benefitting transcutaneous absorption of active substances under the control of the control system 22. Frequencies between 3 MHz and 10 Mhz are believed to be useful in this regard, although this should not be considered limiting. Indeed, possible useful frequencies can range between 20 kHz and above 10 MHz. It would be within the ambit of the skilled person to select appropriate frequency levels and energy intensities so further detail on this point will be omitted for brevity.

As can be seen in FIG. 1, the ultrasonic transducer 20 is positioned such that it emits acoustic energy 23 in a direction towards the working surface 13 such that the acoustic energy travels through and is emitted from the rollable probe head 6. The rollable probe head 6 therefore couples the acoustic energy from the ultrasonic transducer 20 into the working surface 13. More specifically, the ultrasonic transducer 20 is located in the reservoir 10, in a position above the rollable probe head 6 and oriented so that the emission axis E is directed through at least part of the probe head 6. As shown, however, the ultrasonic transducer 20 is positioned so that the emission axis E is vertical and extends through the centre of the probe head 6.

The ultrasonic transducer 20 is supported in the reservoir 10 by a suitable support 28. The support 28 may be a simple strut that holds the transducer in the correct orientation and position within the reservoir 10, although the skilled person would appreciate that other structures would be acceptable.

Beneficially, since the ultrasonic transducer 20 is located in the reservoir 10, it may be at least partially immersed in the flowable substance 11. It is shown here as being fully immersed in the flowable substance 11. The flowable substance therefore acts as a cooling medium for the ultrasonic transducer 20. This means that the run time can be increased without risk of overheating the transducer, or higher power levels may be used.

A benefit of the invention is that the probe 2 is configured such that the rollable probe head 6 performs two functions. Firstly, the probe head 6 is able to roll smoothly across the working surface 13 which reduces friction between the working surface 13 and the probe 2. When used in conjunction with a cosmetic skin product, this provides a more comfortable experience for the user to move the probe 2 about their body, applying suitable pressure where required, thereby also having a massaging effect. Secondly, the probe 2 is configured such that rotation of the rollable probe head 6 dispenses the flowable substance 11 from the reservoir 10 onto the surface of the probe head 6 and therefore onto the working surface 13. This means that a separate source of flowable substance, for example in a squeezable bottle or other dispenser, does not need to be provided. As a result, the flowable product will be used more sparingly and will make less mess.

In the example illustrated in FIG. 1, this is achieved by configuring the housing 4 and the rollable probe head 6 so that is a small gap therebetween so that the flowable substance 11 can be transported out of the opening 12, urged by the motion of the probe head 6. More specifically, it will be appreciated that the receiving section 16 and the probe head 6 are shaped in a complementary way such that a narrow part-spherical gap 30 is defined through which the flowable substance is able to be transported as the probe head 6 rotates. The flowable substance 11 can therefore be considered be transported on the outer surface of the rollable probe head 6 as the surface passes through the reservoir and towards the working surface 13. This can be seen in FIG. 1 which shows the flowable substance 11 adhering to the outer surface of the rollable probe head 6 and being deposited on the working surface.

The size of the gap 30 may be configured to control the movement of the flowable substance. Further, a flow control device may be located in the gap to provide a further means to control the flow. For example, a reticulated foam strip 32 may be located at the top of the receiving section 16, and therefore at the lower end of the reservoir 10 to provide a further resistance to the flowing of the flowable substance. The flow control device may be embodied in different ways, and may take the form of a plurality of discrete channels in the opening 12, for example. This is not essential, however.

Beneficially, the probe 2 may be used to dispense active cosmetic substances on the skin of the user whilst simultaneously applying acoustic energy. Substances that may be used are gels, creams, aqueous solutions and waxes containing a variety of active agents. The range of active ingredients is wide, and can include vitamin substances, caffeine, moisturisers, NSAIDs, analgesics, hair growth promotors, to name a few examples.

In the example in FIG. 1, the ultrasonic transducer 20 is located outside of the rollable probe head 6. The probe head 6 may therefore be a solid ball which may confer advantages in terms of acoustic coupling. The rollable probe head 6 may also be hollow, but containing a couplant fluid to benefit the acoustic coupling between in the transducer 20.

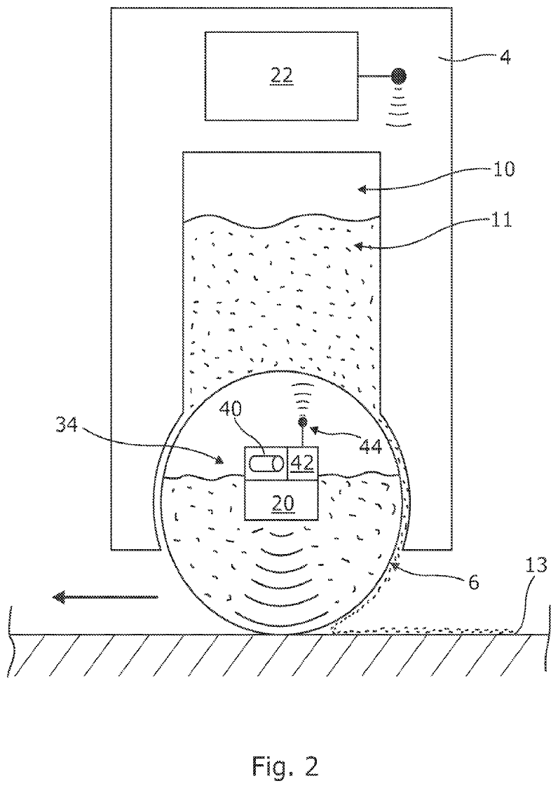

In other examples, the ultrasonic transducer 20 may be located inside the rollable probe head 6. In this respect, reference is made to FIG. 2 in which the same reference numerals will be used to refer to features in common with FIG. 1.

In FIG. 2, the probe 2 is similar to the probe in FIG. 1, although it will be appreciated that the ultrasonic transducer 20 is located inside the rollable probe head 6, which is therefore hollow. Note, however, that the mechanism of transportable of the flowable substance from the reservoir 10 to the working surface remains the same.

Such a configuration has some advantages, one of which is that the structure may be simplified since it is not necessary to provide a support structure and suitable wiring for the transducer in the housing 4 or the reservoir 10. However, it may prove challenging to provide power and control signals to the transducer 20 when it is in the probe head 6, particularly in examples where the probe head 6 is spherical and unconstrained in its axis of rotation.

Therefore, in the example of FIG. 2 the ultrasonic transducer 20 forms part of a transducer package 34. The transducer package 34 provides a self-contained device which provides integrated power and control functions for the ultrasonic transducer 20. As shown in FIG. 2, the transducer package 34 includes the ultrasonic transducer 20, and also a battery module 40 and a transducer controller 42. The transducer controller 42 may be equipped with a suitable wireless communication device 44 for communicating with the control system 22.

In such a configuration, charging of the battery module 40 may be achieved by way of a wireless charging system (not shown). Alternatively, the rollable probe head 6 may be adapted to be disassembled to enable access to the transducer package 34 for recharging purposes.

Various means may be provided to provide support to the ultrasonic transducer 20 inside the rollable probe head 6. As shown in FIG. 2, it is envisaged that the transducer package 34 may be configured so that it is able to be suitably suspended inside a couplant fluid 44 that is contained inside the rollable probe head 6. The couplant fluid 44 therefore serves the purpose of supporting the transmission of acoustic energy emitted from the ultrasonic transducer 20 and also to support the transducer package 34 in an acceptable orientation. Although not shown in FIG. 2, the transducer package 34 may be shaped to ensure that it remains in the correct orientation so that the emitted acoustic energy is always focused in the downwards direction, as shown in the drawings.

Alternatively, or in addition, the transducer package 34 may include an appropriate biasing weight configured so that the ultrasonic transducer 20 stays in the correct orientation.

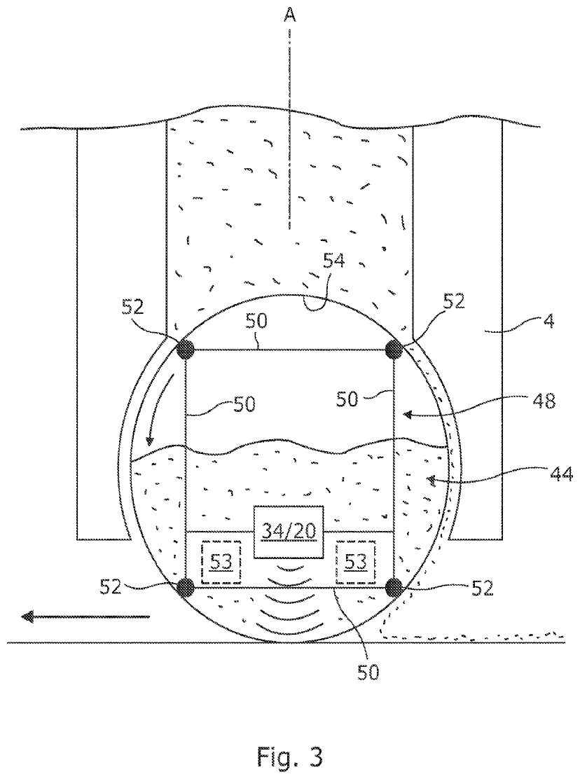

In this example, since the transducer package 34 in effect floats in or on top of the couplant fluid 44 which at least partially fills the rollable probe head 6, movement of the couplant 44 fluid during motion of the probe 2 may cause movement of the transducer package 34 which may affect the transmission of acoustic energy. To mitigate against this, another option for supporting the ultrasonic transducer 20 within the rollable probe head 6 is shown in FIG. 3. Here, it will be appreciated that rather than being supported by floating on the couplant fluid 44, the probe 2 is provided with a gimbal frame 48 that permits the transducer package 34 to move relative to the rollable probe head 6.

The gimbal frame 48 may be embodied in various ways, but here it is shown as taking on a space-frame configuration composed of a network of struts 50. In this way, couplant fluid 44 can flow around the gimbal frame 48 easily.

The gimbal frame 48 is suspended inside the rollable probe head 6 in this example by a plurality of bracing points 52 that bear against an inside surface 54 of the rollable probe head 6 and permit relative movement of the gimbal frame 48. The bracing points 52 are shown defined at junctions of the struts 50, but this is not essential.

In a similar way to the previous examples, the gimbal frame 48 may be suitably weight-biased to ensure that the transducer package 34 remains in the correct orientation despite rotation of the probe head 6. One way in which this may be achieved is for the transducer package 34 to be mounted within the gimbal frame in an off-centre position. One or more suitably positioned biasing masses 53 may also be provided. This would shift the centre of mass of the gimbal frame 48 so that the transducer package 34 tends to pull the gimbal frame 48 into an orientation in which the ultrasonic transducer 20 emits acoustic energy in a direction towards the working surface 7. Here, this direction is aligned with a major axis A of the probe 2.

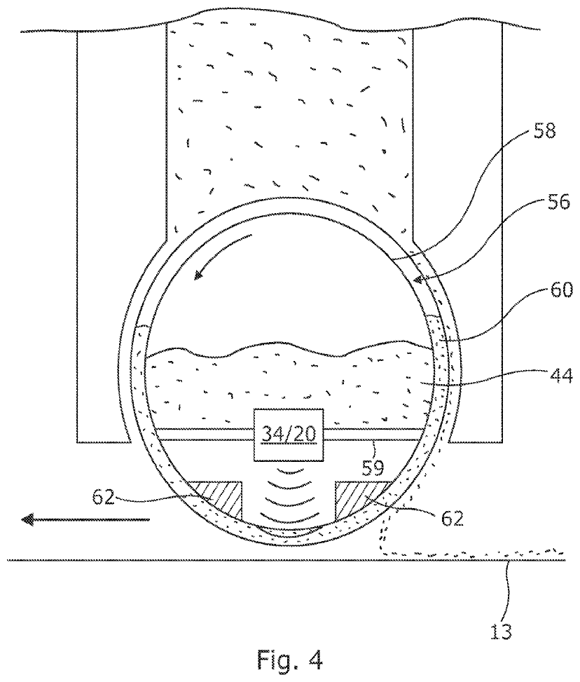

A further example is shown in FIG. 4. In this example, the gimbal frame does not have a space-frame configuration as in the example of FIG. 3 which is braced against the inside of the probe head 6, but instead takes the form of a floating support 56.

The floating support 56 is embodied in the illustrated example by a housing or enclosure 58 within the probe head 6 and which encircles the transducer package 34. The transducer package 34 is supported inside the enclosure 58 by an appropriate support platform 59. In the case where the rollable probe head 6 is spherical in form, the enclosure 58 may also be spherical in shape but with a smaller diameter than the probe head 6, in a manner of nested spheres. In the case where the rollable probe head 6 is cylindrical, the enclosure 58 may be cylindrical in shape but with a small diameter than the probe head 6, in the manner of nested cylinders.

In either of these examples, the difference in size between the enclosure 58 and the inside surface of the rollable probe head 6 means that a space is defined between them. As can be seen, that space is filled with a support fluid 60. The support fluid may be the same fluid as the couplant fluid 44, since it also plays a role in coupling the acoustic energy emitted from the ultrasonic transducer 20 out of the probe head 6 to the working surface 13.

In a similar way to the previous examples, the enclosure 58 may be weight-biased to ensure that the ultrasonic transducer 20 remains oriented correctly. This may be achieved by positioning the transducer package 34 at a relatively low position inside the enclosure, as illustrated, and/or by providing self-righting masses 62 in appropriate positions.

As is shown, the enclosure 58 serves to keep the couplant fluid 44 inside it separate from the support fluid 60 that is trapped between the enclosure 58 and the inside surface of the rollable probe head 6. In this respect, the enclosure 58 is fluid impermeable. In other examples, however, the enclosure 58 may be permeable to fluid.

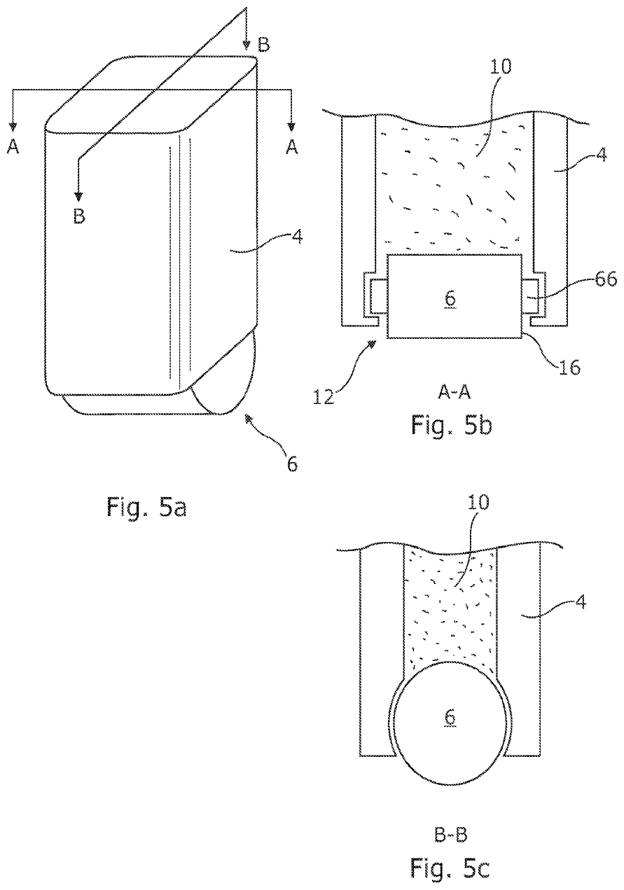

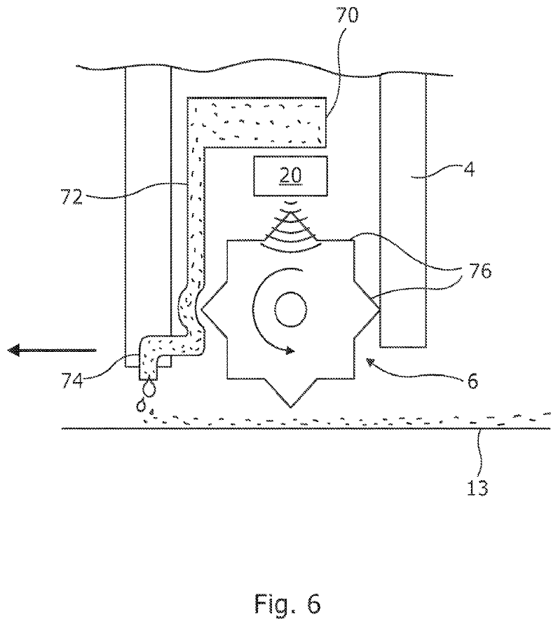

Some variants on the illustrated examples of the invention have already been discussed above. Others will now be described with reference to FIGS. 5a-c and FIG. 6.

In the above examples of the invention, the rollable probe head 6 has been described as being generally spherical as this provides a benefit in terms manoeuvrability of the probe over the skin of a user. As has also been mentioned, the probe head 6 may also be cylindrical in form, like a relatively narrow wheel or a wider barrel shape, and FIGS. 5a-5c provide an illustrated example of this. Here, the rollable probe head 6 is held at a lower end of the probe housing 4. The rollable probe head 6 includes a central axle 66 with which it is rotatably fixed into the receiving section 16 of the opening 12 in the housing 4. The use of the axle mounting of the probe head 6 provides a convenient way to transfer an electrical connection 68 into the probe head 6, as shown in FIG. 5b.

In the above examples of the invention, the rollable probe head 6 is positioned with respect to the reservoir 10 so that a portion of the probe head 6 is exposed to the flowable substance in the reservoir 10 such that rotation of the probe head 6 transports the flowable material from the reservoir 10 to the working surface 12 where it is deposited as the probe 2 is rolled over the surface 7. Although this is a particularly elegant arrangement, other options are possible. One such option is shown in FIG. 6, where features in common with the previous examples are referred to with common reference numbers.

In FIG. 6, the rollable probe head 6 is located at the lower end of the housing 4 as in the previous examples. However, the reservoir 10 in this example is embodied as a tank 70 that is a separate component to the housing 4. The tank 70 is coupled to a dispensing tube 72 that is routed along the inside surface of the housing 4 and which terminates at an outlet or nozzle 74. The routing of the dispensing tube 72 extends past the outer surface of the rollable probe head 6 such that the rollable probe head 6 is able to act on the dispensing tube 72 as the head 6 rotates. For this purpose, the rollable probe head 6 is provided with projections 76 on its outer surface, only two of which are labelled for clarity. The projections 76 may be in the form of a series of ridges that are distributed about the circumference of the rollable probe head and extend parallel to the rolling axis of the probe head 6, for example.

Other forms are acceptable. Due to the proximity of the rollable probe head 6 and the dispensing tube 72, rotation of the probe head 6 causes the projections 76 to rub against the dispensing tube 72. This movement acts as a peristaltic pump, urging flowable substance along the dispensing tube 72 to the nozzle 74 and, from there, to drip onto the working surface 13.

Although the dispensing tube 72 is provided only on one side of the rollable probe head 6 in this example, same or similar arrangements may be provided in other positions around the rollable probe head 6.

The skilled person would appreciate that various modifications could be made to the illustrated examples of the invention without departing from the inventive concept, as defined by the claims.

In the illustrated examples of the invention, the probe 2 has a rollable probe head which is operable to roll across the surface of skin whilst depositing a flowable substance contained within an in-built reservoir. However, in examples of the invention, the probe 2 confers benefits without depositing the flowable substance onto the skin and, instead, a suitable lubricating substance containing the required active ingredient may be applied separately. In such an example, the probe 2 confers benefits over existing cosmetic probes which have simple flat probe heads. One advantage is that the rollable probe head rolls smoothly across the skin and so avoids the ‘dragging’ sensation that is often experienced with conventional flat probe heads. Furthermore, the rolling action of the probe head make it better at following contours of the skin.

1. An ultrasonic probe comprising a rollable probe head and an ultrasonic transducer, wherein the rollable probe head is configured to couple sound energy emitted from the ultrasonic transducer to a working surface, wherein the ultrasonic probe further comprises a reservoir for containing a flowable substance, and wherein the rollable probe head is configured to dispense the flowable substance from the reservoir to the working surface when rotated.

2. The ultrasonic probe of claim 1, wherein the reservoir defines an outlet, and wherein at least part of the rollable probe head is located in the outlet.

3. The ultrasonic probe of claim 2, wherein the outlet is annular in form.

4. The ultrasonic probe of claim 2, wherein the outlet includes a flow control arrangement configured to control transport of the flowable substance through the outlet.

5. The ultrasonic probe of claim 4, wherein the flow control arrangement is a porous strip.

6. The ultrasonic probe of claim 4, wherein the flow control arrangement is a plurality of discrete channels.

7. The ultrasonic probe of claim 1, wherein the ultrasonic transducer is inside the rollable probe head.

8. The ultrasonic probe of claim 7, wherein the ultrasonic transducer is part of a transducer package including an integrated power source.

9. The ultrasonic probe of claim 7, wherein the ultrasonic transducer is supported inside the rollable probe head by a gimbal frame that permits the ultrasonic transducer to move relative to the rollable probe head.

10. The ultrasonic probe of claim 9, wherein the gimbal frame is suspended inside the rollable probe head by a plurality of bracing points that bear against an inside surface of the rollable probe head and permit relative movement of the gimbal frame.

11. The ultrasonic probe of claim 9, wherein the gimbal frame is supported inside the rollable probe head by a fluid support.

12. The ultrasonic probe of claim 11, wherein the gimbal frame includes a housing encircling the transducer and which defines a fluid-filled gap between the housing and the probe head.

13. The ultrasonic probe of claim 9, wherein the gimbal frame is self-righting.

14. The ultrasonic probe of claim 7, wherein the rollable probe head contains couplant fluid to facilitate transmission of energy emitted from the ultrasonic transducer out of the rollable probe head.

15. The ultrasonic probe of claim 14, wherein the ultrasonic transducer is at least partially immersed in the couplant fluid such that the ultrasonic transducer is cooled by the couplant fluid.

16. The ultrasonic probe of claim 1, wherein the ultrasonic transducer is located remote from the rollable probe head.

17. The ultrasonic probe of claim 16, wherein the rollable probe head is located between the working surface and the ultrasonic transducer.

18. The ultrasonic probe of claim 15, wherein the ultrasonic transducer is located in the reservoir such that it is immersible in the flowable substance.

19. An ultrasonic cosmetic probe for applying acoustic energy to skin, the ultrasonic cosmetic probe comprising a rollable probe head and an ultrasonic transducer, wherein the rollable probe head is configured to couple sound energy emitted from the ultrasonic transducer to a skin surface.
